(19) United States
(12) Patent Application Publication
(10) Pub. No.: US 2025/0128044 A1
(43) Pub. Date: Apr. 24, 2025

(54) BILATERAL MECHANICAL CIRCULATORY SUPPORT SYSTEM

(71) Applicant: Abiomed, Inc., Danvers, MA (US)

(72) Inventors: Zhenghong Tao, Danvers, MA (US); Michael Finnegan, Danvers, MA (US); Daniel Podlisny, Danvers, MA (US)

(73) Assignee: Abiomed, Inc., Danvers, MA (US)

(21) Appl. No.: 18/922,770

(22) Filed: Oct. 22, 2024

Related U.S. Application Data
(60) Provisional application No. 63/592,524, filed on Oct. 23, 2023.

(51) Int. Cl. A61M 60/157; A61M 60/216; A61M 60/47

(52) U.S. Cl. CPC A61M 60/157; A61M 60/216; A61M 60/47

(57) ABSTRACT

A bilateral mechanical circulatory support system is described. The bilateral mechanical circulatory support system includes a first heart pump configured to be positioned across an aortic valve of a patient, the first heart pump including a first pressure sensor and a second pressure sensor, a second heart pump configured to be positioned across a pulmonary valve of the patient, the second heart pump including a third pressure sensor and a fourth pressure sensor, and at least one controller. The at least one controller is configured to control operation of the first heart pump and the second heart pump, the at least one controller configured to receive pressure signals from the first pressure sensor, the second pressure sensor, the third pressure sensor, and the fourth pressure sensor. The system further includes a display configured to display one or more waveforms or values based, at least in part, on the pressure signals.

### RELATED APPLICATIONS

This application claims the benefit under 35 U.S.C. § 119 (c) to U.S. Provisional Patent Application No. 63/592,524, filed Oct. 23, 2023, and titled, “BILATERAL MECHANICAL CIRCULATORY SUPPORT SYSTEM,” the entire contents of which is incorporated by reference herein.

### FIELD OF THE INVENTION

This disclosure relates to a bilateral mechanical circulatory support system.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella® family of devices (Abiomed, Inc., Danvers, MA).

### SUMMARY

More than 35% of patients with left-side heart dysfunctions tend to develop right-side heart dysfunctions due, for example, to imbalanced blood flow. To this end, some embodiments of the present disclosure relate to a bilateral mechanical circulatory support (MCS) system that includes right-and left-side heart pumps, which can be controlled independently as needed to, for example, provide balanced blood flow within the patient's heart. Additionally, each of the right-and left-side heart pumps may include multiple pressure sensors (e.g., optical pressure sensors) that enable measurements of the pressure within the chambers of the patient's heart during operation of the bilateral MCS system. One or more waveforms and/or values based on the pressure signals sensed from the pressure sensors may be displayed to a healthcare provider to enable the healthcare provider to make informed decisions about the care of a patient while the patient is using the bilateral MCS system. For instance, the healthcare provider may adjust the pump speed of one or both of the heart pumps to provide a more balanced flow of blood through the patient's heart based, at least in part, on viewing the one or more waveforms and/or values associated with the pressure signals.

In some embodiments, a bilateral mechanical circulatory support system is provided. The bilateral mechanical circulatory support system includes a first heart pump configured to be positioned across an aortic valve of a patient, the first heart pump including a first pressure sensor and a second pressure sensor, a second heart pump configured to be positioned across a pulmonary valve of the patient, the second heart pump including a third pressure sensor and a fourth pressure sensor, and at least one controller configured to control operation of the first heart pump and the second heart pump, the at least one controller configured to receive pressure signals from the first pressure sensor, the second pressure sensor, the third pressure sensor, and the fourth pressure sensor. The system further includes a display configured to display one or more waveforms or values based, at least in part, on the pressure signals.

In one aspect, the at least one controller includes a first controller configured to control operation of the first heart pump and receive pressure signals from the first pressure sensor and the second pressure sensor, and a second controller configured to control operation of the second heart pump and receive pressure signals from the third pressure sensor and the fourth pressure sensor. In another aspect, the system further includes an auxiliary device communicatively coupled to the at least one controller via one or more networks, wherein the display is associated with the auxiliary device. In another aspect, the first pressure sensor is configured to sense an aortic pressure and the second pressure sensor is configured to sense a left ventricular pressure when the first heart pump is positioned across the aortic valve of the patient. In another aspect, the third pressure sensor is configured to sense a pulmonary artery pressure and the fourth pressure sensor is configured to sense a superior vena cava pressure or an inferior vena cava pressure when the second heart pump is positioned across the pulmonary valve of the patient. In another aspect, the display is configured to simultaneously display a waveform and/or value for each of the aortic pressure, left ventricular pressure, pulmonary artery pressure and superior vena cava pressure or inferior vena cava pressure.

In another aspect, the system further includes at least one processor configured to derive one or more cardiac metrics based, at least in part, on two or more of the pressure signals, and the display is configured to display the one or more cardiac metrics. In another aspect, the one or more cardiac metrics includes a left differential pressure and/or a right differential pressure. In another aspect, deriving one or more cardiac metrics based, at least in part, on two or more of the pressure signals includes deriving at least one cardiac metric based, at least in part, on all of the pressure signals. In another aspect, the pressure signals includes a first pressure signal, a second pressure signal, a third pressure signal, and a first pressure signal, and deriving one or more cardiac metrics includes determining based, at least in part, on the first pressure signal and/or the second pressure signal, a first rate of blood flow through the aortic valve, and determining based, at least in part, on the third pressure signal and/or the fourth pressure signal, a second rate of blood flow through the pulmonary valve, and the display is configured to display an indication of blood flow based, at least in part, on the first rate and the second rate. In another aspect, displaying an indication of blood flow includes displaying a comparison of blood flow between the aortic valve and the pulmonary valve of the patient. In another aspect, displaying an indication of blood flow includes displaying an alert when the first rate and the second rate differ by more than a threshold amount. In another aspect, the at least one controller is configured to adjust a pump speed of the first heart pump and/or the second heart pump based, at least in part, on the first rate and the second rate. In another aspect, adjusting the pump speed of the first heart pump and/or second heart pump is performed when a difference between the first rate and the second rate exceeds a threshold amount.

In another aspect, the system further includes at least one processor configured to simulate a cardiac function of the patient based, at least in part, on two or more of the pressure signals and an adjusted pump speed of the first heart pump and/or the second heart pump, and the display is configured to display the simulated cardiac function. In another aspect, the display includes a user interface that enables a healthcare provider to specify the adjusted pump speed for the first heart pump and/or the second heart pump for use in the simulation of the cardiac function of the patient. In another aspect, simulating a cardiac function of the patient includes simulating the cardiac function based, at least in part, on all of the pressure signals and the adjusted pump speed of the first heart pump and/or the second heart pump.

In some embodiments, an auxiliary device for a bilateral mechanical circulatory support system is provided. The auxiliary device includes a communications interface configured to receive pressure information from at least one controller configured to control operation of a first heart pump and a second heart pump. The first heart pump is configured to be positioned across an aortic valve of a patient, the first heart pump including a first pressure sensor and a second pressure sensor and the second heart pump is configured to be positioned across a pulmonary valve of the patient, the second heart pump including a third pressure sensor and a fourth pressure sensor. The auxiliary device further includes a display configured to display one or more waveforms or values based, at least in part, on the received pressure information.

In one aspect, the first pressure sensor is configured to sense an aortic pressure and the second pressure sensor is configured to sense a left ventricular pressure when the first heart pump is positioned across the aortic valve of the patient. In another aspect, the third pressure sensor is configured to sense a pulmonary artery pressure and the fourth pressure sensor is configured to sense a superior vena cava pressure or an inferior vena cava pressure when the second heart pump is positioned across the pulmonary valve of the patient. In another aspect, the display is configured to simultaneously display a waveform and/or value for each of the aortic pressure, left ventricular pressure, pulmonary artery pressure and superior vena cava pressure or inferior vena cava pressure.

In another aspect, the auxiliary device further includes at least one processor configured to derive one or more cardiac metrics based, at least in part, on the pressure information, and the display is configured to display the one or more cardiac metrics. In another aspect, the one or more cardiac metrics includes a left differential pressure and/or a right differential pressure. In another aspect, deriving one or more cardiac metrics based, at least in part, on the pressure information includes deriving at least one cardiac metric based, at least in part, on all pressure signals included in the pressure information. In another aspect, the pressure information includes a first pressure signal, a second pressure signal, a third pressure signal, and a fourth pressure signal, and deriving one or more cardiac metrics includes determining based, at least in part, on the first pressure signal and/or the second pressure signal, a first rate of blood flow through the aortic valve, and determining based, at least in part, on the third pressure signal and/or the fourth pressure signal, a second rate of blood flow through the pulmonary valve, wherein the display is configured to display an indication of blood flow based, at least in part, on the first rate and the second rate. In another aspect, displaying an indication of blood flow includes displaying a comparison of blood flow between the aortic valve and the pulmonary valve of the patient. In another aspect, displaying an indication of blood flow includes displaying an alert when the first rate and the second rate differ by more than a threshold amount.

In another aspect, the auxiliary device further includes at least one processor configured to simulate a cardiac function of the patient based, at least in part, on the pressure information and an adjusted pump speed of the first heart pump and/or the second heart pump, and the display is configured to display the simulated cardiac function. In another aspect, the display includes a user interface that enables a healthcare provider to specify the adjusted pump speed for the first heart pump and/or the second heart pump for use in the simulation of the cardiac function of the patient. In another aspect, simulating a cardiac function of the patient includes simulating the cardiac function based, at least in part, on all pressure signals included in the pressure information and the adjusted pump speed of the first heart pump and/or the second heart pump.

In some embodiments, a method of monitoring a patient's cardiac function using a bilateral mechanical circulatory support device is provided. The method includes sensing, using a first pressure sensor and a second pressure sensor located on a first heart pump positioned across an aortic valve of a patient, a respective first pressure signal and a second pressure signal, sensing, using a third pressure sensor and a fourth pressure sensor located on a second heart pump positioned across a pulmonary valve of the patient, a respective third pressure signal and a fourth pressure signal, and displaying on a display communicatively coupled to the first heart pump and the second heart pump via at least one controller, one or more waveforms or values based, at least in part, on the first pressure signal, the second pressure signal, the third pressure signal, and the fourth pressure signal.

In one aspect, the first pressure sensor is configured to sense an aortic pressure and the second pressure sensor is configured to sense a left ventricular pressure when the first heart pump is positioned across the aortic valve of the patient. In another aspect, the third pressure sensor is configured to sense a pulmonary artery pressure and the fourth pressure sensor is configured to sense a superior vena cava pressure or an inferior vena cava pressure when the second heart pump is positioned across the pulmonary valve of the patient. In another aspect, displaying one or more waveforms or values includes simultaneously displaying a waveform and/or value for each of the aortic pressure, left ventricular pressure, pulmonary artery pressure and superior vena cava pressure or inferior vena cava pressure. In another aspect, the method further includes deriving one or more cardiac metrics based, at least in part, on two or more of the first, second, third and fourth pressure signals, and displaying on the display, the one or more cardiac metrics. In another aspect, the one or more cardiac metrics includes a left differential pressure and/or a right differential pressure. In another aspect, deriving one or more cardiac metrics based, at least in part, on two or more of the first, second, third and fourth pressure signals includes deriving at least one cardiac metric based, at least in part, on all of the first, second, third, and fourth pressure signals. In another aspect, deriving one or more cardiac metrics includes determining based, at least in part, on the first pressure signal and/or the second pressure signal, a first rate of blood flow through the aortic valve, and determining based, at least in part, on the third pressure signal and/or the fourth pressure signal, a second rate of blood flow through the pulmonary valve, wherein displaying one or more waveforms or values includes displaying an indication of blood flow based, at least in part, on the first rate and the second rate. In another aspect, displaying an indication of blood flow includes displaying a comparison of blood flow between the aortic valve and the pulmonary valve of the patient. In another aspect, displaying an indication of blood flow includes displaying an alert when the first rate and the second rate differ by more than a threshold amount. In another aspect, the method further includes adjusting a pump speed of the first heart pump and/or the second heart pump based, at least in part, on the first rate and the second rate. In another aspect, adjusting the pump speed of the first heart pump and/or second heart pump is performed when a difference between the first rate and the second rate exceeds a threshold amount.

In another aspect, the method further includes simulating a cardiac function of the patient based, at least in part, on two or more of the first, second, third and fourth pressure signals and an adjusted pump speed of the first heart pump and/or the second heart pump, and displaying one or more waveforms or values includes displaying the simulated cardiac function. In another aspect, the method further includes receiving via user interface displayed on the display, the adjusted pump speed for the first heart pump and/or the second heart pump for use in the simulation of the cardiac function of the patient. In another aspect, simulating a cardiac function of the patient includes simulating the cardiac function based, at least in part, on all of the first, second, third and fourth pressure signals and the adjusted pump speed of the first heart pump and/or the second heart pump.

### BRIEF DESCRIPTION OF DRAWINGS

FIG. 1A illustrates a pump system in accordance with some embodiments of the present disclosure.

FIG. 1B is a cross-sectional view of a portion of the pump system of FIG. 1A.

FIG. 2 illustrates a pump system in accordance with some embodiments of the present disclosure.

FIG. 3 illustrates a pump system in accordance with some embodiments of the present disclosure.

FIG. 4 illustrates a pump system with a multi-channel pressure sensor in accordance with some embodiments of the present disclosure.

FIG. 5 is a block diagram of components of a bilateral mechanical circulatory support (MCS) system in accordance with some embodiments of the present disclosure.

FIG. 6 illustrates a process for displaying pressure information for a bilateral MCS system in accordance with some embodiments of the present disclosure.

### DETAILED DESCRIPTION

A bilateral mechanical circulatory support (MCS) system that includes both a left-side intracardiac blood pump and a right-side intracardiac blood pump may provide benefits to some patients with diminished cardiac function or who are at risk of developing both left-side and right-side cardiac dysfunction. To this end, some embodiments of the present disclosure relate to a bilateral MCS system that includes independently-controllable left-side and right-side heart pumps, which may enable a healthcare provider flexibility in managing the support provided to the patient during the course of their treatment when compared with systems that only include a heart pump inserted on one side of the patient's heart. In some embodiments, one or both of the left-side and right-side heart pumps includes multiple pressure sensors such that the pressure at both the inlet and outlet of the heart pump can be measured directly. One or more cardiac metrics based on the sensed pressure signals may be displayed to a healthcare provider to facilitate care management for the patient within which the bilateral MCS system is used.

To provide an overall understanding of the systems, methods, and devices described herein, certain illustrative examples will be described. Although various examples may describe specific medical procedures and/or uses of intracardiac blood pumps, it will be understood that the technology described herein may be employed in any suitable context.

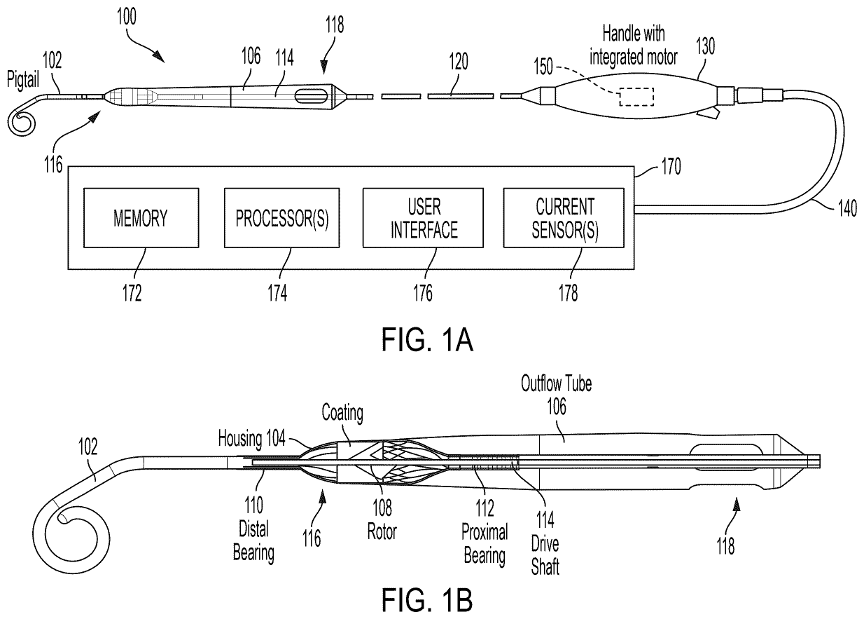

A pump system 100 (also referred to herein simply as pump 100) for use with some embodiments of the present disclosure is shown in FIGS. 1A and 1B. As shown, pump 100 may be coupled to a control unit 170. Pump 100 may include a distal atraumatic tip 102, a pump housing 104 surrounding a rotor 108, an outflow tube 106, distal bearing 110, proximal bearing 112, inlet 116, outlet 118, catheter 120, handle 130, cable 140, and motor 150. As will be appreciated, although shown with an atraumatic tip, in some embodiments, the pump may not include such a tip. Pump housing 104 may be configured as a frame structure formed by a mesh with openings which may, at least in part, be covered by an elastic material. As will be appreciated, although shown as a frame structure, in some embodiments, the pump housing may be solid. A proximal portion of pump housing 104 may extend into and be mounted in the hollow interior of outflow tube 106, and a distal portion of pump housing 104 may extend distally beyond the distal end of outflow tube 106. The exposed openings in the pump housing 104 extending distally beyond outflow tube 106 may form the inlet 116 of pump 100. The proximal end of outflow tube 106 may include a plurality of openings that form the outlet 118 of pump 100. Rotor 108 may be rotationally mounted between distal bearing 110 and proximal bearing 112, and may be coupled to a distal end of drive shaft 114. Drive shaft 114 may be flexible and may extend through catheter 120, through the hollow interior of outflow tube 106, into handle 130 and is coupled to motor 150, which is housed in handle 130. The proximal end of handle 130 may be coupled via cable 140 to control unit 170. A fluid may be circulated through the catheter 120 proximate to the drive shaft 114 and in the space surrounding the distal bearing 110 and proximal bearing 112 to lubricate those components and reduce friction during operation of the pump 100.

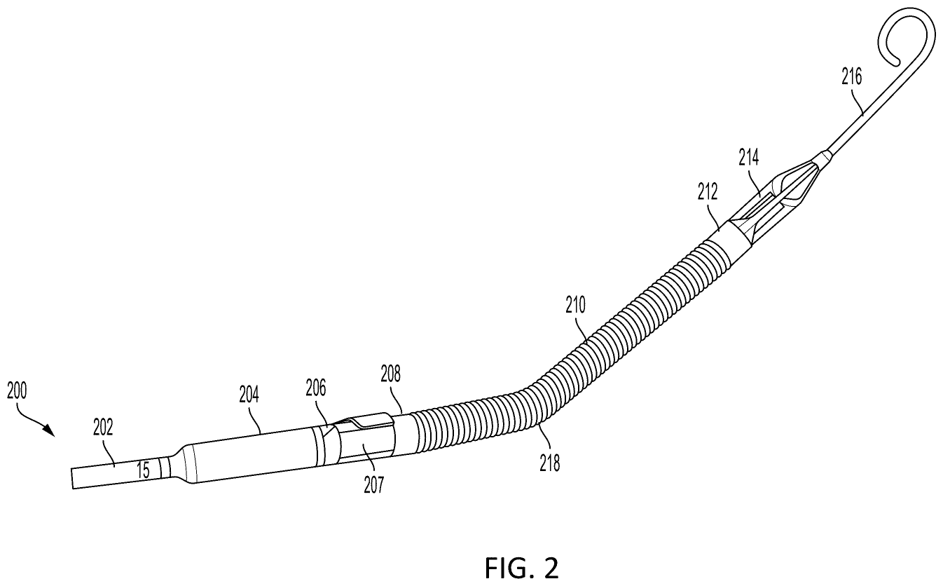

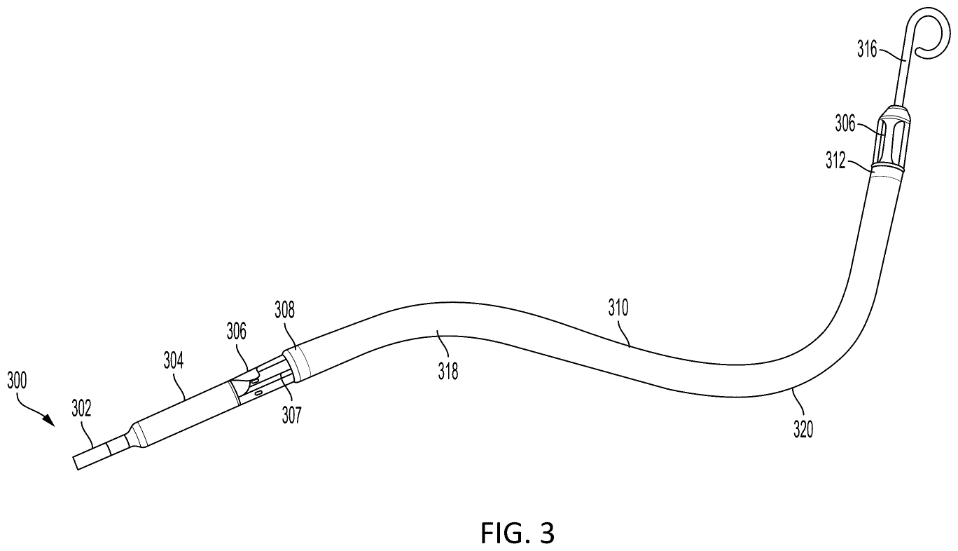

Control unit 170 may include one or more memory 172, one or more processors 174, a user interface 176, and one or more sensors, such as current sensors 178. Processor(s) 174 may comprise one or more microcontrollers, one or more microprocessors, one or more application specific integrated circuits (ASICs), one or more digital signal processors, program memory, or other computing components. Processor(s) 174 may be communicatively coupled to the other components (e.g., memory 172, user interface 176, current sensor(s) 178) of control unit 170 and may be configured to control one or more operations of pump 100. As will be appreciated, although control unit 170 is shown connected to pump 100, control unit may be connected to intracardiac blood pump assembly 200 or intracardiac blood pump assembly 300 described in connection with FIGS. 2 and 3. As a non-limiting example, control unit 170 may be implemented as an Automated Impella Controller™ from ABIOMED, Inc., Danvers, MA. In some aspects, memory 172 is included as a portion of processor(s) 174 rather than being provided as a separate component.

During operation, processor(s) 174 may be configured to control the electrical power delivered to motor 150 (e.g., by controlling a power supply (not shown)) by a power supply line (not shown) in cable 140, thereby controlling the speed of the motor 150. Current sensor(s) 178 may be configured to sense motor current associated with an operating state of the motor 150, and processor(s) 174 may be configured to receive the output of current sensor(s) 408 as a motor current signal. Processor(s) 174 may further be configured to determine a flow through the pump 100 based, at least in part, on the motor current signal and the motor speed, as described in more detail herein. Current sensor(s) 178 may be included in control unit 170 or may be located along any portion of the power supply line in cable 140. Additionally or alternatively, current sensor(s) 178 may be included in motor 150 and processor(s) 174 may be configured to receive the motor current signal via a data line (not shown) in cable 140 coupled to processor(s) 174 and motor 150.

Memory 172 may be configured to store computer-readable instructions and other information for various functions of the components of control unit 170. In one aspect, memory 172 includes volatile and/or non-volatile memory, such as, an electrically erasable programmable read-only memory (EEPROM).

User interface 176 may be configured to receive user input via one or more buttons, switches, knobs, etc. Additionally, user interface 176 may include a display configured to display information and one or more indicators, such as light indicators, audio indicators, etc., for conveying information and/or providing alerts regarding the operation of pump 100.

Pump 100 may be designed to be insertable into a patient's body, e.g., into a left ventricle of the heart, such as via an introducer system. Although some of the systems and/or methods disclosed herein are described for modulating a pump speed of a pump inserted into the left ventricle of a heart, it should be appreciated that the systems and/or methods described herein may also be applied to other types of ventricular support systems, such as a ventricular support system inserted into the right ventricle of the heart. In one aspect, housing 104, rotor 108, and outflow tube 106 may be radially compressible to enable pump 100 to achieve a relatively small outer diameter of, for example, 9 Fr (3 mm) during insertion. When pump 100 is inserted into the patient, e.g., into a left ventricle, handle 130 and motor 150 may remain disposed outside the patient. As will be appreciated, in other embodiments, the motor of the pump system may be disposed inside the patient upon insertion. During operation, motor 150 is controlled by processor(s) 404 to drive rotation of drive shaft 114 and rotor 108 to convey blood from inlet 116 to outlet 118. It is to be appreciated that rotor 108 may be rotated by motor 150 in reverse to convey blood in the opposite direction (in this case, the openings of 118 form the inlet and the openings of 116 form the outlet). In one aspect, pump 100 may be configured to be used for weeks to months to years to support the heart function of a patient with chronic heart failure, though it should be understood that the technology described herein is not limited to any particular types of procedures and/or use durations.

A blood pump system for use with some embodiments is shown in FIG. 2. FIG. 2 depicts an exemplary intracardiac blood pump assembly 200 adapted for left heart support, in accordance with aspects of the disclosure. As shown in FIG. 2, an intracardiac blood pump assembly adapted for left heart support may include an elongate catheter 202, a motor 204, a cannula 210, a blood inflow cage 214 arranged at or near the distal end 212 of the cannula 210, a blood outflow cage 206 arranged at or near the proximal end 208 of the cannula 210, and an optional atraumatic extension 216 arranged at the distal end of the blood inflow cage 214.

In some aspects of the technology, motor 204 may be configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood outflow cage 206, for example, within the proximal end 208 of the cannula 210 or within a pump housing 207 coupled to the proximal end 208 of the cannula 210. In some aspects of the technology, rather than the impeller being driven by an onboard motor 204, the impeller may instead be coupled to an elongate drive shaft (or drive cable) which is driven by a motor located external to the patient.

Catheter 202 may house electrical lines coupling the motor 204 to one or more electrical controllers and/or sensors. Alternatively, where the impeller is driven by an external motor, an elongate drive shaft may pass through catheter 202. Catheter 202 may also include a purge fluid conduit, a lumen configured to receive a guidewire, etc.

The blood inflow cage 214 may include one or more apertures or openings configured to allow blood to be drawn into cannula 210 when the motor 204 is operating. Likewise, blood outflow cage 206 may include one or more apertures or openings configured to allow blood to flow from the cannula 210 out of the intracardiac blood pump assembly 200. Blood inflow cage 214 and outflow cage 206 may be composed of any suitable bio-compatible material(s). For example, blood inflow cage 214 and/or blood outflow cage 206 may be formed out of bio-compatible metals such as stainless steel, titanium, or biocompatible polymers such as polyurethane. In addition, the surfaces of blood inflow cage 214 and/or blood outflow cage 206 may be treated in various ways, including, but not limited to etching, texturing, or coating or plating with another material. For example, the surfaces of blood inflow cage 214 and/or blood outflow cage 206 may be laser textured.

Cannula 210 may include a flexible hose portion. For example, cannula 210 may be composed, at least in part, of a polyurethane material. In addition, cannula 210 may include a shape-memory material. For example, cannula 210 may comprise a combination of a polyurethane material and one or more strands or coils of a shape-memory material such as Nitinol. Cannula 210 may be formed such that it includes one or more bends or curves in its relaxed state, or it may be configured to be straight in its relaxed state. In that regard, as shown in the exemplary arrangement of FIG. 2, the cannula 210 may have a single pre-formed anatomical bend 218 based on the portion of the left heart in which it is intended to operate. Despite this bend 218, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bend 218 once the shape-memory material is exposed to the heat of a patient's body.

Atraumatic extension 216 may assist with stabilizing and positioning the intracardiac blood pump assembly 200 in the correct position in the patient's heart. Atraumatic extension 216 may be solid or tubular. If tubular, atraumatic extension 216 may be configured to allow a guidewire to be passed through it to further assist in the positioning of the intracardiac blood pump assembly 200. Atraumatic extension 216 may be any suitable size. For example, atraumatic extension 216 may have an outer diameter in the range of 4-8 Fr. Atraumatic extension 216 may be composed, at least in part, of a flexible material, and may be any suitable shape or configuration such as a straight configuration, a partially curved configuration, a pigtail-shaped configuration as shown in the example of FIG. 2, etc. Atraumatic extension 216 may also have sections with different stiffnesses. For example, atraumatic extension 216 may include a proximal section that is stiff enough to prevent it from buckling, thereby keeping the blood inflow cage 214 in the desired location, and a distal section that is softer and has a lower stiffness, thereby providing an atraumatic tip for contact with a wall of the patient's heart and to allow for guidewire loading. In such a case, the proximal and distal sections of the atraumatic extension 216 may be composed of different materials, or may be composed of the same material with the proximal and distal sections being treated to provide different stiffnesses.

Notwithstanding the foregoing, as mentioned above, atraumatic extension 216 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As described herein, the intracardiac blood pump assembly 200 may be inserted percutaneously. For example, when used for left heart support, intracardiac blood pump assembly 200 may be inserted via a catheterization procedure through the femoral artery or axillary artery, into the aorta, across the aortic valve, and into the left ventricle. Once positioned in this way, the intracardiac blood pump assembly 200 may deliver blood from the blood inflow cage 214, which sits inside the left ventricle, through cannula 210, to the blood outflow cage 206, which sits inside the ascending aorta. In some aspects of the technology, intracardiac blood pump assembly 200 may be configured such that bend 218 will rest against a predetermined portion of the patient's heart when the intracardiac blood pump assembly 200 is in a desired location. Likewise, the atraumatic extension 216 may be configured such that it rests against a different predetermined portion of the patient's heart when the intracardiac blood pump assembly 200 is in the desired location.

FIG. 3 depicts an exemplary intracardiac blood pump assembly 300 adapted for right heart support, in accordance with aspects of the disclosure. As shown in FIG. 3, an intracardiac blood pump assembly adapted for right heart support may include an elongate catheter 302, a motor 304, a cannula 310, a blood inflow cage 314 arranged at or near the proximal end 308 of the cannula 310, a blood outflow cage 306 arranged at or near the distal end 312 of the cannula 310, and an optional atraumatic extension 316 arranged at the distal end of the blood outflow cage 306.

As with the exemplary assembly of FIG. 2, motor 304 may be configured to rotatably drive an impeller (not shown), thereby generating suction sufficient to draw blood into cannula 310 through the blood inflow cage 314, and to expel the blood out of cannula 310 through the blood outflow cage 306. In that regard, the impeller may be positioned distal of the blood inflow cage 314, for example, within the proximal end 308 of the cannula 310 or within a pump housing 307 coupled to the proximal end 308 of the cannula 310. Here as well, in some aspects of the technology, rather than the impeller being driven by an onboard motor 304, the impeller may instead be coupled to an elongate drive shaft (or drive cable) which is driven by a motor located external to the patient.

The cannula 310 of FIG. 3 may serve the same purpose, and may have the same properties and features described above with respect to cannula 210 of FIG. 2. However, as shown in the exemplary arrangement of FIG. 3, the cannula 310 may have two pre-formed anatomical bends 318 and 320 based on the portion of the right heart in which it is intended to operate. Here again, despite the existence of bends 318 and 320, the cannula 310 may nevertheless also be flexible, and may thus be capable of straightening (e.g., during insertion over a guidewire), or bending further (e.g., in a patient whose anatomy has tighter dimensions). Further in that regard, cannula 310 may include a shape-memory material configured to allow the cannula 310 to be a different shape (e.g., straight or mostly straight) at room temperatures, and to form bends 318 and/or 320 once the shape-memory material is exposed to the heat of a patient's body.

The catheter 302 and atraumatic extension 316 of FIG. 3 may serve the same purpose and may have the same properties and features described above with respect to catheter 202 and atraumatic extension 216 of FIG. 2. Likewise, other than being located at opposite ends of the cannula from those of FIG. 2, the blood inflow cage 314 and blood outflow cage 306 of FIG. 3 may be similar to the blood inflow cage 214 and blood outflow cage 206 of FIG. 2, and thus may have the same properties and features described above.

Like the exemplary assembly of FIG. 2, the intracardiac blood pump assembly 300 of FIG. 3 may also be inserted percutaneously. For example, when used for right heart support, intracardiac blood pump assembly 300 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 300 may deliver blood from the blood inflow cage 314, which sits inside the inferior vena cava, through cannula 310, to the blood outflow cage 306, which sits inside the pulmonary artery.

A heart pump (e.g., pump 100, intracardiac blood pump assembly 200, intracardiac blood pump assembly 300, etc.) may include a pressure sensor (e.g., an optical pressure sensor) configured to detect a pressure within the aorta of a patient's heart when the heart pump is properly positioned in the left side of the heart or detect a pressure within the pulmonary artery of the patient's heart when the heart pump is properly positioned in the right side of the heart. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate through the heart pump. The differential pressure across the aortic valve (for a left-side device) or pulmonary valve (for a right-side device) may also indirectly be determined based on the pressure signal measuring the pressure in the aorta (for a left-side device) or the pulmonary artery (for a right-side device) and the set of stored values.

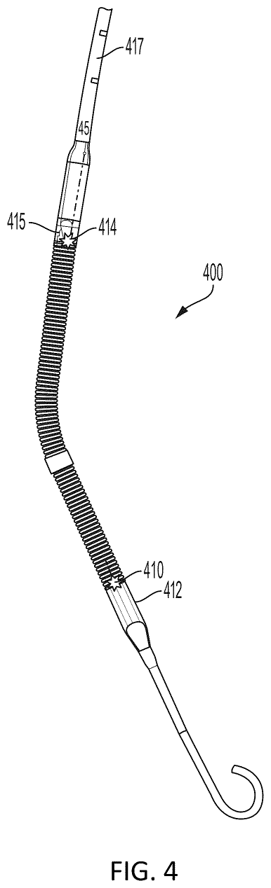

In some embodiments, a heart pump may include multiple pressure sensors (e.g., multiple optical pressure sensors). For example, a left-side heart pump may include pressure sensors to directly sense pressure in both the aorta and the left ventricle rather than having to infer the pressure in the left ventricle based on the pressure sensor signal sensed in the aorta, as discussed above. Similarly, a right-side heart pump may include pressure sensors to directly sense pressure in both the pulmonary artery and the right ventricle rather than having to infer the pressure in the right ventricle based on the pressure sensor signals sensed in the pulmonary artery. Use of multiple pressure sensors may also be referred to as implementing a multi-channel pressure sensor. FIG. 4 illustrates an embodiment of heart pump 400 including multiple pressure sensors, for use with some embodiments of the present disclosure. Heart pump 400 includes a first pressure sensor 410 arranged near an inlet area 412 and a second pressure sensor 414 arranged near an output area 415. When properly positioned within the heart of a patient, the second pressure sensor 410 may be configured to measure a pressure sensor signal used to determine a left/right ventricular blood pressure. In such implementations, sensor cables (e.g., optical fibers) may be disposed within catheter tube 417 to provide a connection between each of the first pressure sensor 410 and the second pressure sensor 414 and the controller.

The inventors have recognized and appreciated that to accommodate multiple pressure sensors within a heart pump it may be useful to provide a lower power and/or smaller sensor than pressure sensors used in some conventional heart pumps. Additionally, the use of optical-based pressure sensors may have advantages over electronic or other types of pressure sensors, including, but not limited to, their smaller size, their small or negligible pressure drift and their durability.

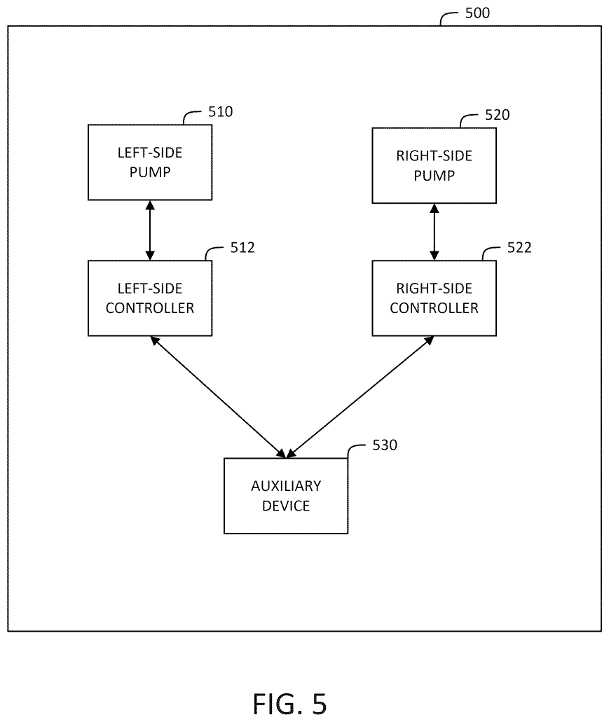

The inventors have also recognized and appreciated that some patients may benefit from a bilateral heart pump system that may be able to fully offload the patient's heart. To this end, some embodiments of the present disclosure relate to a bilateral system that includes right-and left-side heart pumps, which can be controlled independently as needed to, for example, provide balanced blood flow through a patient's heart. FIG. 5 illustrates a bilateral mechanical circulatory support (MCS) system 500 in accordance with some embodiments of the present disclosure. Bilateral MCS system 500 includes a left-side pump 510 configured to be positioned across the aortic valve of a patient's heart to provide left heart support and a right-side pump 520 configured to be positioned across the pulmonary valve of the patient's heart to provide right-heart support.

Each of the left-side pump 510 and the right-side pump 520 may be individually controllable. For instance, as shown in FIG. 5, left-side pump 510 may be communicatively coupled to left-side controller 512 and right-side pump 520 may be communicatively coupled to right-side controller 522. Left-side controller 512 may be configured to send control commands to left-side pump 510 to control operation of the motor in the pump (e.g., by adjusting the motor current and accordingly, the pump speed of pump 510). Left-side controller 512 may also be configured to receive sensor information (e.g., pressure sensor information) from the left-side pump 510. The received sensor information may be processed and/or displayed by left-side controller 512. Similarly, right-side controller 522 may be configured to send control commands to right-side pump 520 to control operation of the motor in the pump (e.g., by adjusting the motor current and accordingly, the pump speed of right-side pump 520). Right-side controller 522 may also be configured to receive sensor information (e.g., pressure sensor information) from the right-side pump 520. The received sensor information may be processed and/or displayed by right-side controller 522.

As shown in FIG. 5, bilateral MCS system 500 may also include auxiliary device 530 communicatively coupled to each of left-side controller 512 and right-side controller 522. Auxiliary device 530 may include a display configured to display one or more waveforms and/or values based on information received from the left-side controller 512 and the right-side controller 522. For instance, each of left-side controller 512 and right-side controller 522 may be configured to receive pressure signal information from pressure sensor(s) arranged on left-side pump 510 and right-side pump 520, respectively, and auxiliary device 530 may be configured to display one or more waveforms and/or values based on the pressure signal information. For example, one or more pressure signal waveforms and/or values (e.g., values averaged or otherwise considered over a time window) from each of the left-side controller 512 and the right side controller 522 may be displayed on auxiliary device 530. In some embodiments of the present disclosure, one or more cardiac metrics (e.g., left differential pressure, right differential pressure, right-side blood flow, left-side blood flow, bilateral blood flow imbalance, etc.) may be calculated based, at least in part, on at least two of the sensed pressure signals, and the calculated cardiac metric(s) may be displayed on auxiliary device 530. In some embodiments of the present disclosure, the auxiliary device 530 may be configured to display an alert based on one or more of the calculated cardiac measures. For instance, when a rate of blood flow on the right side and the left side of the patient's heart differ by more than a particular amount representative of a blood flow imbalance, the auxiliary device 530 may display an alert indicating the detected blood flow imbalance. A healthcare provider observing the alert may then interact with the bilateral MCS system to adjust a pump speed of one or both of left-side pump 510 or right-side pump 520 to address the imbalance.

In some embodiments, one or both of left-side pump 510 and right-side pump 520 may include multiple pressure sensors (e.g., multiple optical pressure sensors), and the pressure information provided by the left-side controller 512 and the right-side controller 522 to auxiliary device 530 may be displayed as one or more waveforms and/or values. For instance, when both left-side pump 510 and right-side pump 520 include multiple pressure sensors, the pressure information may include a left ventricular (LV) pressure signal, an aorta (AO) pressure signal, a pulmonary artery (PA) pressure signal, and a superior vena cava (SVC) or inferior vena cava (IVC) pressure signal. Auxiliary device 530 may be configured to display one or more waveforms and/or values based on these pressure signals to describe the functioning of the patient's heart. Pressure waveforms and/or values based directly on the sensed pressure signals and/or heart functional parameters derived from the sensed pressure signals may be displayed by auxiliary device 530. As an example, one or more waveforms and/or values for each of LV pressure, AO pressure, PA pressure and SVC or IVC pressure may be simultaneously displayed by auxiliary device 530. Providing a physician or other healthcare provider with such information on a single display device may enable the healthcare provider to assess the treatment status of the patient and/or make adjustments to the amount of support provided by the bilateral MCS system 500 (e.g., to balance the blood flow across both sides of the heart). Additionally, some embodiments of the present disclosure may provide diagnostic information about the patient's heart in a way that reduces the number of devices used to monitor the heart's performance compared with some existing systems.

In some embodiments, auxiliary device 530 may be communicatively coupled to left-side controller 512 and right-side controller 522 via one or more networks (e.g., the Internet). In some embodiments auxiliary device 530 enables a physician or other healthcare provider with remote access to information sensed by the left-side pump 510 and the right-side pump 520 for remote monitoring and/or control of left-side pump 510 and right-side pump 520.

Although FIG. 5 shows bilateral MCS system 500 including multiple controllers (i.e., left-side controller 512, right-side controller 522, and optionally auxiliary device 530), in some embodiments bilateral MCS system 500 may include a single controller (or any suitable number of controllers) configured to control the left-side pump 510 and right-side pump 520.

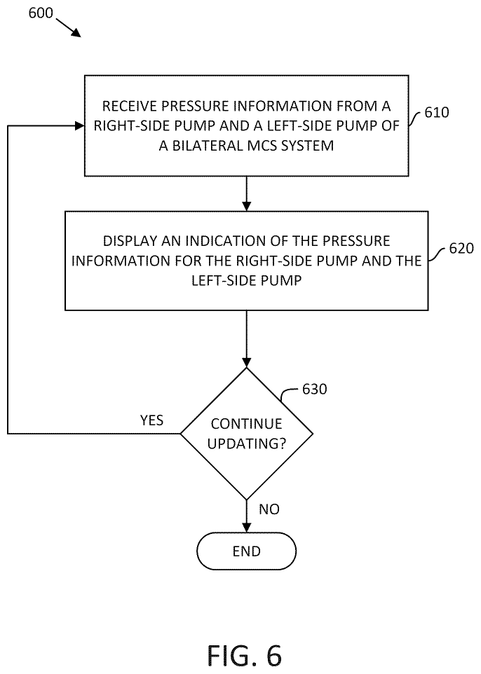

FIG. 6 is a flowchart of a process 600 for displaying pressure information for a bilateral mechanical circulatory support (MCS) system in accordance with some embodiments of the present disclosure. Process 600 may begin in act 610, where pressure information from a right-side pump and a left-side pump of the bilateral MCS system is received. For instance, the pressure information may be received by an auxiliary device in communication with respective controllers of the right-side pump and left-side pump. In some embodiments, the pressure information may include direct measurements of the aortic pressure and the left ventricular pressure from the left-side pump, and the pulmonary artery pressure and superior vena cava or inferior vena cava pressure from the right-side pump. Process 600 may then proceed to act 620, where an indication of the pressure information associated with measurements from the right-side pump and the left side pump are simultaneously displayed to enable a physician or other healthcare provider the ability to have a more complete view of the patient's cardiac function while being supported by the bilateral MCS system. Providing such information to the healthcare provider may facilitate patient management by enabling the healthcare provider to more easily determine that adjustments to the operation of the right-side pump and/or the left-side pump may be needed to improve the patient's care (e.g., by balancing the blood flow through both sides of the patient's heart). Process 600 may then proceed to act 630, where it is determined whether to continue updating the display of the pressure information. If it is determined in act 630 to continue updating the display, process 600 may then return to act 610, where additional pressure information is received from the right-side pump and the left-side pump of the bilateral MCS device. If it is determined in act 630 that updating the display is no longer desired, process 600 may end.

In some embodiments of the present disclosure, a healthcare provider may interact with a user interface (e.g., displayed on an auxiliary device such as auxiliary device 530) to simulate how adjustments in pump speed of the left heart pump and/or the right heart pump affect cardiac function of the patient. For instance, information associated with pump operation (e.g., pump speed) and physiological parameters associated with the patient (e.g., based on the sensed pressure signals) within whom a bilateral MCS device is implanted may be displayed on the user interface. In some embodiments, the user interface may include a recommendation to adjust one or both of the pump speeds of the left-side and right-side pumps. Prior to adjusting the pump speed(s) (e.g., based on the recommendation), the healthcare provider may simulate the cardiac function of the patient based on the sensed pressure signals from the dual pressure sensors on one or both of the implanted pumps in the bilateral MCS system.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A bilateral mechanical circulatory support system, comprising:
a first heart pump configured to be positioned across an aortic valve of a patient, the first heart pump including a first pressure sensor and a second pressure sensor;
a second heart pump configured to be positioned across a pulmonary valve of the patient, the second heart pump including a third pressure sensor and a fourth pressure sensor;
at least one controller configured to control operation of the first heart pump and the second heart pump, the at least one controller configured to receive pressure signals from the first pressure sensor, the second pressure sensor, the third pressure sensor, and the fourth pressure sensor; and
a display configured to display one or more waveforms or values based, at least in part, on the pressure signals.

2. The system of claim 1, wherein the at least one controller includes:
a first controller configured to control operation of the first heart pump and receive pressure signals from the first pressure sensor and the second pressure sensor; and
a second controller configured to control operation of the second heart pump and receive pressure signals from the third pressure sensor and the fourth pressure sensor.

3. The system of claim 1, further comprising:
an auxiliary device communicatively coupled to the at least one controller via one or more networks, wherein the display is associated with the auxiliary device.

4. The system of claim 1, wherein the first pressure sensor is configured to sense an aortic pressure and the second pressure sensor is configured to sense a left ventricular pressure when the first heart pump is positioned across the aortic valve of the patient.

5. The system of claim 4, wherein the third pressure sensor is configured to sense a pulmonary artery pressure and the fourth pressure sensor is configured to sense a superior vena cava pressure or an inferior vena cava pressure when the second heart pump is positioned across the pulmonary valve of the patient.

6. The system of claim 5, wherein the display is configured to simultaneously display a waveform and/or value for each of the aortic pressure, left ventricular pressure, pulmonary artery pressure and superior vena cava pressure or inferior vena cava pressure.

7. The system of claim 1, further comprising:
at least one processor configured to derive one or more cardiac metrics based, at least in part, on two or more of the pressure signals,
wherein the display is configured to display the one or more cardiac metrics.

8. (canceled)

9. (canceled)

10. The system of claim 7, wherein the pressure signals include a first pressure signal, a second pressure signal, a third pressure signal, and fourth pressure signal, and wherein deriving one or more cardiac metrics comprises:
determining based, at least in part, on the first pressure signal and/or the second pressure signal, a first rate of blood flow through the aortic valve; and
determining based, at least in part, on the third pressure signal and/or the fourth pressure signal, a second rate of blood flow through the pulmonary valve,
wherein the display is configured to display an indication of blood flow based, at least in part, on the first rate and the second rate.

11. The system of claim 10, wherein displaying an indication of blood flow comprises displaying a comparison of blood flow between the aortic valve and the pulmonary valve of the patient.

12. The system of claim 11, wherein displaying an indication of blood flow comprises displaying an alert when the first rate and the second rate differ by more than a threshold amount.

13. The system of claim 10, wherein the at least one controller is configured to adjust a pump speed of the first heart pump and/or the second heart pump based, at least in part, on the first rate and the second rate.

14. (canceled)

15. The system of claim 1, further comprising:
at least one processor configured to simulate a cardiac function of the patient based, at least in part, on two or more of the pressure signals and an adjusted pump speed of the first heart pump and/or the second heart pump,
wherein the display is configured to display the simulated cardiac function.

16. The system of claim 15, wherein the display includes a user interface that enables a healthcare provider to specify the adjusted pump speed for the first heart pump and/or the second heart pump for use in the simulation of the cardiac function of the patient.

17. The system of claim 15, wherein simulating a cardiac function of the patient comprises simulating the cardiac function based, at least in part, on all of the pressure signals and the adjusted pump speed of the first heart pump and/or the second heart pump.

18. An auxiliary device for a bilateral mechanical circulatory support system, the auxiliary device comprising:
a communications interface configured to receive pressure information from at least one controller configured to control operation of a first heart pump and a second heart pump, wherein
the first heart pump is configured to be positioned across an aortic valve of a patient, the first heart pump including a first pressure sensor and a second pressure sensor, and
the second heart pump is configured to be positioned across a pulmonary valve of the patient, the second heart pump including a third pressure sensor and a fourth pressure sensor; and
a display configured to display one or more waveforms or values based, at least in part, on the received pressure information.

19-21. (canceled)

22. The auxiliary device of claim 18, further comprising:
at least one processor configured to derive one or more cardiac metrics based, at least in part, on the pressure information,
wherein the display is configured to display the one or more cardiac metrics.

23. (canceled)

24. (canceled)

25. The auxiliary device of claim 22, wherein the pressure information includes a first pressure signal, a second pressure signal, a third pressure signal, and a fourth pressure signal, and wherein deriving one or more cardiac metrics comprises:
determining based, at least in part, on the first pressure signal and/or the second pressure signal, a first rate of blood flow through the aortic valve; and
determining based, at least in part, on the third pressure signal and/or the fourth pressure signal, a second rate of blood flow through the pulmonary valve,
wherein the display is configured to display an indication of blood flow based, at least in part, on the first rate and the second rate.

26. The auxiliary device of claim 25, wherein displaying an indication of blood flow comprises displaying a comparison of blood flow between the aortic valve and the pulmonary valve of the patient.

27. (canceled)

28. The auxiliary device of claim 18, further comprising:
at least one processor configured to simulate a cardiac function of the patient based, at least in part, on the pressure information and an adjusted pump speed of the first heart pump and/or the second heart pump,
wherein the display is configured to display the simulated cardiac function.

29-30. (canceled)

31. A method of monitoring a patient's cardiac function using a bilateral mechanical circulatory support device, the method comprising:
sensing, using a first pressure sensor and a second pressure sensor located on a first heart pump positioned across an aortic valve of a patient, a respective first pressure signal and a second pressure signal;
sensing, using a third pressure sensor and a fourth pressure sensor located on a second heart pump positioned across a pulmonary valve of the patient, a respective third pressure signal and a fourth pressure signal; and
displaying on a display communicatively coupled to the first heart pump and the second heart pump via at least one controller, one or more waveforms or values based, at least in part, on the first pressure signal, the second pressure signal, the third pressure signal, and the fourth pressure signal.

32-45. (canceled)
